(19) United States
(12) Patent Application Publication
(10) Pub. No.: US 2010/0136123 A1
(43) Pub. Date: Jun. 3, 2010

(54) Microcapsule Sheet

(75) Inventors: Shinya Izumida, Shiga (JP); Takashi Iwade, Shiga (JP); Takashi Nagayama, Shiga (JP); Masashi Motoi, Shiga (JP); Kiyohito Yamamoto, Shiga (JP)

Correspondence Address: GLOBAL IP COUNSELORS, LLP, 1233 20TH STREET, NW, SUITE 700, WASHINGTON, DC, 20036-2680, US

(73) Assignee: TORAY ENGINEERING CO., LTD., Tokyo (JP)

(21) Appl. No.: 12/445,250

(22) PCT Filed: Oct. 12, 2007
(86) PCT No.: PCT/JP2007/069913

§ 371 Date: Jan. 7, 2010

(30) Foreign Application Priority Data
Oct. 12, 2006 (JP) 2006-278541

(51) Int. Cl. A61K 9/50

(52) U.S. Cl. 424/490

(57) ABSTRACT

In order to heighten the density of microcapsules and facilitate handling by the unit composed of microcapsules in a given amount smaller than in the whole sheet, a microcapsule sheet which comprises a substrate constituted of an edible film and microcapsules each obtained by surrounding a core layer with a first shell film and a second shell film, the microcapsules being arranged according to a given pattern so as to be arranged only in those regions of the substrate which are separated from each other.

### TECHNICAL FIELD

The present invention relates to a microcapsule sheet which is manufactured by arranging microcapsules on a substrate.

### BACKGROUND ART

From the past, it is proposed that a microcapsule sheet is manufactured by forming a base bottom layer, a core layer, and a surface layer on a substrate (refer to a patent document 1, and a patent document 2).

Patent Document 1 International Publication WO 2001/89486A1

Patent Document 2 Japanese Patent Laid Open Publication 2002-531394

### DISCLOSURE OF THE INVENTION

### Problems to be Solved by the Invention

The microcapsule disclosed in the patent document 1, and the patent document 2 is heightened its density in general. When the density is heightened, the interval between. the microcapsules becomes an extremely narrow interval, and a most close-placed array pattern is employed as an array pattern.

When the microcapsules are formed in a most close-placed array, it is impossible that microcapsules in a required amount are separated by linearly cutting.

Therefore, handling of microcapsules on only substrate basis is possible. It is substantially impossible that microcapsules are handled in an arbitrary amount basis.

When microcapsules are applied for medical drug application, it becomes necessary that microcapsules are separated from a substrate, the separated microcapsules are collected, and the collected microcapsules are housed within an edible case. Therefore, entire working becomes complicated.

The present invention was made in view of the above problems. It is an object of the present invention to provide a microcapsule sheet which can be used for medical drug application without the work for separating microcapsules from a substrate and for collecting the separated microcapsules.

### Means for Solving the Problems

A microcapsule sheet according to the present invention is a microcapsule sheet manufactured by determining or virtually determining multiple regions on a substrate constituted of edible film, and by arranging microcapsules within each of the multiple regions.

It is preferable that the microcapsule is made by surrounding a core layer with a first shell film and a second shell film.

It is also preferable that the multiple regions are multiple regions each leaving space between the neighboring region. It is further preferable that each region is a region within which a predetermined number of microcapsules are arranged.

### Effects of the Invention

The microcapsule sheet according to the present invention heightens the density of microcapsules because microcapsules are arranged within each region with high arranging density, and facilitates handling of microcapsules in a predetermined amount basis by easily cutting and separating the substrate between regions, the predetermined amount being less than the amount of microcapsules within the whole microcapsule sheet.

Also, the microcapsules are taken in as they are without separating microcapsules from a substrate. Therefore, manufacturing work can extremely be simplified when the microcapsules are for medical drug application, because works for separating microcapsules, collecting separated microcapsules, packing collected microcapsules, and the like becomes unnecessary. Of course, manufacturing cost can be reduced because lowering of yield in separating work, collecting work, packing work, and the like is not realized.

Further, it is possible to encapsulate on microcapsule sheet basis. It is also possible to fix a microcapsule sheet into an easy taking shape. It is further possible to roll a microcapsule sheet and to fill the rolled microcapsule sheet into a capsule. Therefore, manufacturing cost is reduced because microcapsules can be handled on microcapsule sheet basis without handling of microcapsules directly.

When the multiple regions are multiple regions spaced apart from one another, and each region is a region within which a predetermined number of microcapsules are arranged, desired amount of microcapsules can be taken on region basis. For example, when the region is determined to include an amount of drug determined based upon body weight or taking amount at once, convenience is further improved.

### BEST MODE FOR CARRYING OUT THE INVENTION

Hereinafter, referring to the attached drawings, we explain embodiments of a microcapsule sheet according to the present invention in detail.

FIG. 1 is a schematic view illustrating an apparatus of an example for manufacturing a microcapsule sheet according to the present invention.

This apparatus comprises an X-Y stage 2 for supporting a substrate 1 constituted of edible film and for moving the substrate 1 two dimensionally, a nozzle apparatus 3 having three liquid-drop-supplying nozzles for supplying a liquid drop containing first shell material, a liquid drop containing core material, and a liquid drop containing second shell material from an above position with respect to the substrate 1, respectively, and for adhering the supplied liquid drop to the surface of the substrate 1, and a drying apparatus 4 for drying the adhered liquid drop so as to form a first shell film, and a second shell film.

Each of the liquid-drop-supplying nozzles discharges a liquid drop using a piezoelectric head which discharges a liquid drop, utilizing the distorting characteristic of piezoelectric ceramics, or a thermal ink jet head which discharges a liquid drop utilizing thermal energy, for example, like an ink jet nozzle used in an ink jet printer. Therefore, a discharge amount can be controlled with high accuracy.

The nozzle apparatus 3 is composed to move to and fro corresponding to the disposition of the three liquid-drop-supplying nozzles so as to operate the three liquid-drop-supplying nozzles at a same position sequentially, because the nozzle apparatus 3 has three liquid-drop-supplying nozzles.

As the drying apparatus 4, an infra-red heater, for example, can be employed.

Next, operation of the apparatus illustrated in FIG. 1 is described with reference to FIG. 2.

The substrate 1 is moved to a predetermined position by the X-Y stage 2. A liquid drop containing first shell material is adhered to the substrate 1 by operating one of the three liquid-drop-supplying nozzles of the nozzle apparatus 3. The adhered liquid drop has an almost hemisphere shape under this condition (refer to FIG. 2(A)).

Then, the drying apparatus 4 is operated. The surface of the liquid drop is dried so that a film is formed, at first. Drying operation is continued thereafter, then solvent and the like within the liquid drop is evaporated so that the film formed at first becomes concave shaped film (refer to FIG. 2(B)). However, the film formed at first may be simply flattened depending upon species of shell material, liquid drop, and the like.

Thereafter, another one of the three liquid-drop-supplying nozzles of the nozzle apparatus 3 is operated so that a liquid drop containing core material is adhered onto the first shell film The adhered liquid drop is dried by the drying apparatus 4 so that a core layer is formed (refer to FIG. 2C).

Thereafter, a further one of the three liquid-drop-supplying nozzles of the nozzle apparatus 3 is operated so that a liquid drop containing second shell material is adhered to cover the core layer. Then, the liquid drop containing second shell material is dried by operating the drying apparatus 4 so that a second shell film is formed (refer to FIG. 2(D)).

By the above operations, microcapsules 5 are manufactured on the substrate 1 constituted of edible film.

Further, microcapsules 5 can be arranged by a predetermined pattern only within a desired region on the substrate 1, because the substrate 1 can be moved by the X-Y stage 2. Specifically, the substrate 1 constituted of edible film is virtually partitioned into multiple regions 1a, and a separating part 1b is set between neighboring regions 1a, and microcapsules 5 are arranged only within each region 1a, as is illustrated in FIG. 3, for example. In addition, virtually partitioning into multiple regions 1a, and setting of a separating part 1b between neighboring regions 1a can be realized by setting a number, size, arranging pattern and the like of the region 1a, and width of the separating part 1b in a controller (not illustrated) for controlling the X-Y stage 2, for example. It is preferable that the size of the region 1a is determined based upon a number of microcapsules 5 to be arranged within the region 1a. In this case, total amount of liquid drops containing core material per region 1a is determined to be a predetermined amount.

It is preferable that the pattern of microcapsules 5 arranged within each region 1a is most close-placed array. In this case, the density of microcapsules 5 arranged within each region 1a is heightened. It is also preferable that the width of the separating part 1b is determined to be a width which permits easy cutting with scissors or the like. It is further preferable that perforation for cutting is formed at a central section in width direction of the separating part 1b. In this case, one region 1a can easily be separated from other region 1a without using scissors.

Medical drug is exemplified as the use application of a microcapsule sheet manufactured by the above manner.

A starch film, a polysaccharide film, or a protein film can be employed as the edible film, an enteric polymer film, for example, can be employed as the first shell material, and insulin, erythropoietin, peptide or the like can be employed as the core material, because the microcapsule sheet is used as medical drug. Gel forming material may be added to the core material as needed. Further, water insoluble polymer, or wax can be employed as the second shell material.

It is preferable that material on which microcapsule forming face water-repellent pattern is formed, is employed as the substrate 1 constituted of edible film. In this case, wetting and expanding are prevented from occurrence so that material liquid for forming the first shell material can be existed within the water-repellent pattern. Consequently, the microcapsule 5 can be miniaturized by downsizing the water-repellent pattern.

For example, wetting and expanding of a microcapsule forming face is prevented from occurrence so that the microcapsule 5 having a small diameter can be formed, by applying water-repellent processing to faces (shaded portions in FIG. 4) other than microcapsule forming faces, as is illustrated in FIG. 4.

Also, wetting and expanding of a microcapsule forming face is prevented from occurrence so that the microcapsule 5 can be miniaturized, by patterning water-repellent frame (having a ring shape, lattice shape or the like) around the microcapsule forming face, as is illustrated in FIGS. 5 and 6.

It is preferable that material which has formed a concave sections at microcapsule forming faces by embossing, is employed as the substrate 1 constituted of edible film. In this case, material for forming first shell material can be applied within the concave sections by a printing method using a squeegee.

### INDUSTRIAL APPLICABILITY

The present invention provides a microcapsule sheet which heightens the density of microcapsules 5, and makes handling easy to handle microcapsules on a predetermined amount basis which amount is less than the total amount of microcapsules within the microcapsule sheet.

### BRIEF DESCRIPTION OF THE DRAWINGS

FIG. 1 is a schematic view illustrating an apparatus of an example for manufacturing a microcapsule sheet according to the present invention;

FIG. 2 are schematic views useful in understanding manufacturing process of a microcapsule for manufacturing on a substrate;

FIG. 3 is a schematic view illustrating a disposition of microcapsules of an example on a microcapsule sheet;

FIG. 4 is a schematic plan view illustrating a substrate of an example which is applied water-repellent processing for surrounding microcapsule forming faces;

FIG. 5 is a schematic plan view illustrating a substrate of another example which is applied water-repellent processing for surrounding microcapsule forming faces; and

FIG. 6 is a schematic plan view illustrating a substrate of a further example which is applied water-repellent processing for surrounding microcapsule forming faces.

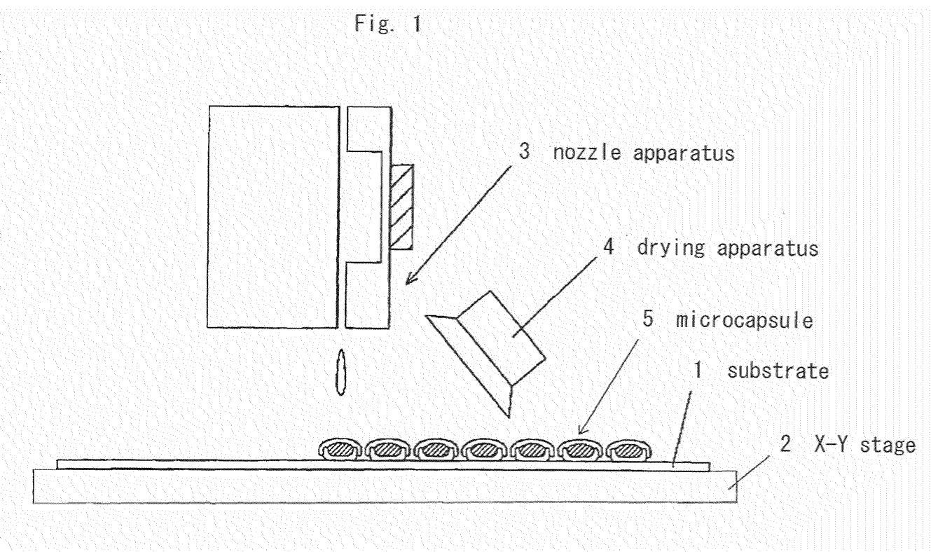

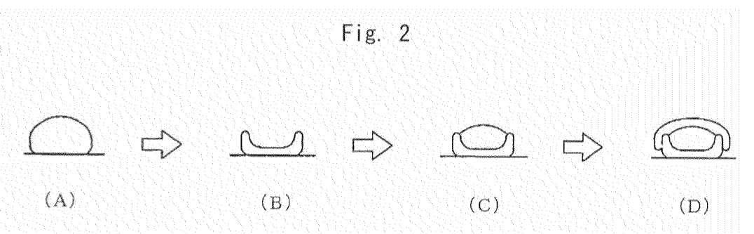

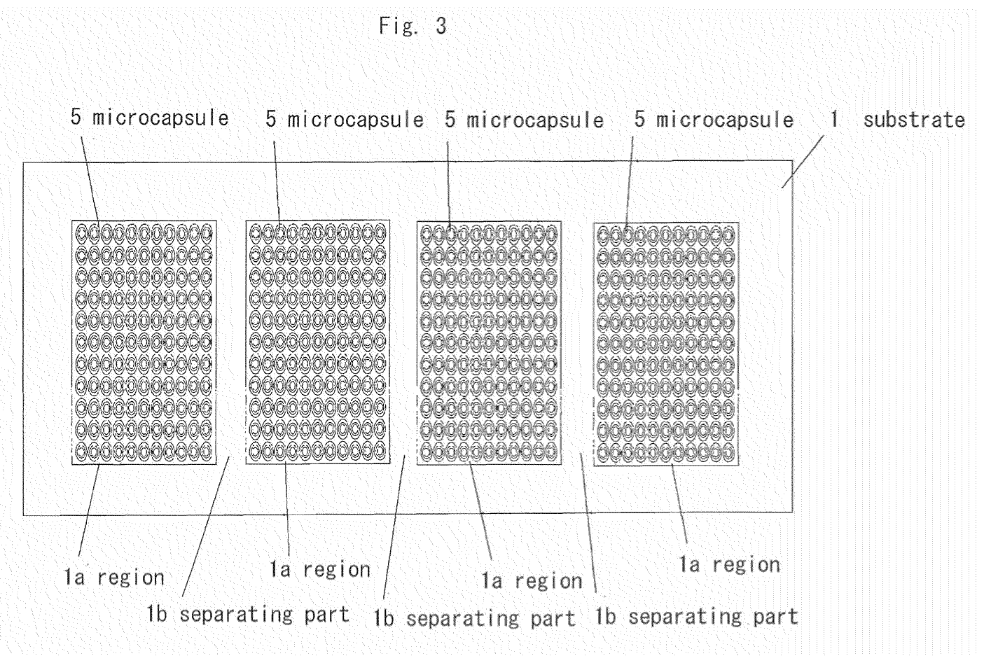

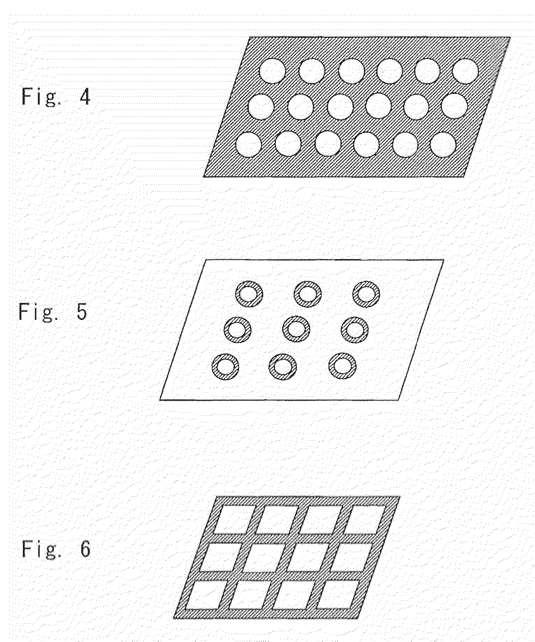

### DESCRIPTION OF REFERENCES

1 substrate
1a region
1b separating part
2 X-Y stage
3 nozzle apparatus
4 drying apparatus
5 microcapsule

What is claimed is:

1. A microcapsule sheet comprising;
a substrate constituted of edible film, the substrate being virtually divided into multiple regions; and
microcapsules arranged within each region.

2. A microcapsule sheet as set forth in claim 1, wherein the microcapsule is manufactured by surrounding a core layer with a first shell film and a second shell film.

3. A microcapsule sheet as set forth in claim 1, wherein the multiple regions are multiple regions each of which is spaced apart from a neighboring region, and wherein each region is a region where a predetermined number of microcapsules are arranged.

4. A microcapsule sheet as set forth in claim 2, wherein the multiple regions are multiple regions each of which is spaced apart from a neighboring region, and wherein each region is a region where a predetermined number of microcapsules are arranged.
